(19) United States
(12) Patent Application Publication
(10) Pub. No.: US 2018/0320223 A1
(43) Pub. Date: Nov. 8, 2018

(54) IDED DOUBLE-STRANDED PROBES FOR DETECTION OF NUCLEIC ACID AND USES OF SAME

(71) Applicant: QuanDx Inc., San Jose, CA (US)

(72) Inventors: Xiaojun LEI, San Jose, CA (US); Yuan YUAN, San Jose, CA (US); Guo-liang YU, Hillsborough, CA (US); Qiang LI, San Jose, CA (US)

(21) Appl. No.: 15/559,827

(22) PCT Filed: Mar. 20, 2016
(86) PCT No.: PCT/US2016/023333

§ 371 Date: Sep. 19, 2017

Related U.S. Application Data
(60) Provisional application No. 62/135,644, filed on Mar. 19, 2015.

(51) Int. Cl. C12Q 1/6818

(52) U.S. Cl. CPC C12Q 1/6818

(57) ABSTRACT

The present disclosure provides a method for detecting multiple target nucleic acid sequences in a sample using a plurality of IDed double-stranded probes. Each IDed double-stranded probe comprises a double-stranded nucleic acid hybridization probe associated with an IDed substrate. Also provided is a method for determining the sequence of a nucleic acid by using a plurality of IDed double-stranded probes.

### CROSS-REFERENCE TO RELATED APPLICATIONS

This application claims priority to U.S. provisional patent application No. 62/135,644, filed Mar. 19, 2015, the disclosure of which is incorporated herein by reference.

### FIELD OF THE INVENTION

The present invention generally relates to probes for detection of nucleic acids.

### BACKGROUND OF THE INVENTION

A hybridization probe is a fragment of DNA or RNA labeled with molecular marker, e.g., radioactive or fluorescent molecules, used in the DNA or RNA samples to detect the presence of nucleotide sequences that are complementary to the sequence of the probe. Traditionally, the use of hybridization probe in detecting the presence of nucleotide sequences, e.g., in Southern or Northern blot, requires separation of hybridized and unhybridized probes, which is complex and time-consuming. Recently, an array of new hybridization probes, e.g., S′-exonuclease (TaqMan™) probe, molecular beacons, fluorescence energy transfer probes, Scorpion probes, have been developed to provide more rapid, simple and quantitative detection. However, the probes mentioned above are difficult to design and synthesize, and have limited specificity. On the other hand, although it has been a long-existing desire to develop multiplex detection method by using multiple hybridization probes to detect the presence of multiple nucleotide sequences in one reaction, the method is limited by the number of molecular markers available to be used together.

Therefore, there is a continuing need to develop new hybridization probes and method thereof for multiplex detection of nucleotide sequences.

### BRIEF SUMMARY OF THE INVENTION

In one aspect, the present disclosure provides a composition comprising a double-stranded nucleic acid hybridization probe associated with an IDed substrate. In certain embodiments, the double-stranded nucleic acid hybridization probe consists of (i) a first oligonucleotide comprising a first sequence complementary to a target sequence; (ii) a second oligonucleotide comprising a second sequence that is complementary to the first sequence but is shorter than the first sequence by up to ten nucleotides; (iii) a fluorophore linked to one of the first and second oligonucleotide; and (iv) a fluorophore quencher linked to the other of the first and second oligonucleotide, wherein the fluorophore quencher quenches the fluorophore when the first oligonucleotide hybridizes to the second oligonucleotide; and (b) an IDed substrate associated with the double-stranded nucleic acid hybridization probe.

In certain embodiments, said first oligonucleotide is capable of spontaneously hybridizing to the target sequence in the presence of the second oligonucleotide. In certain embodiments, the first oligonucleotide is not capable of spontaneously hybridizing to a mismatched sequence that differs from the target sequence by a single nucleotide substitution. In certain embodiments, the free energy released by hybridization of the first and second oligonucleotides is less than the free energy released by hybridization of the first oligonucleotide to the target sequence but greater than the free energy released by hybridization of the first oligonucleotide to a mismatched sequence that differs from the target sequence by a single nucleotide substitution.

In certain embodiments, the oligonucleotide described above can comprise one or more nucleotide analogs (e.g., altered backbone, sugar, or nucleobase). In certain embodiments, the nucleotide analog is selected from the group consisting of 5-bromouracil, a peptide nucleic acid nucleotide, a xeno nucleic acid nucleotide, a morpholino, a locked nucleic acid nucleotide, a glycol nucleic acid nucleotide, a threose nucleic acid nucleotide, a dideoxynucleotide, a cordycepin, a 7-deaza-GTP, a fluorophore (e.g. rhodamine or flurescein linked to the sugar), a thiol containing nucleotide, a biotin linked nucleotide, a fluorescent base analog, a methyl-7-guanosine, a methylated nucleotide, an inosine, thiouridine, a pseudourdine, a dihydrouridine, a queuosine, and a wyosine. In certain embodiments, the nucleotide analog is a locked nucleic acid nucleotide.

In certain embodiments, the first and second oligonucleotides hybridize to produce a double-stranded blunt end, and wherein the fluorophore and the quencher are linked to the blunt end.

In certain embodiments, the target sequence has a length of 5˜20 nucleotides. In certain embodiments, the second oligonucleotide is shorter than the first sequence by 1, 2, 3, 4, 5, 6, 7, 8, 9 or 10 nucleotides. In certain embodiments, the second oligonucleotide is shorter than the first sequence by 1 to 5 nucleotides. In certain embodiments, the second oligonucleotide is shorter than the first sequence by 2 to 7 nucleotides. In certain embodiments, the second oligonucleotide is shorter than the first sequence by 3 to 8 nucleotides. In certain embodiments, the second oligonucleotide is shorter than the first sequence by 4 to 9 nucleotides. In certain embodiments, the second oligonucleotide is shorter than the first sequence by 5 to 10 nucleotides. In certain embodiments, the first sequence is 100% complementary to the target sequence.

In certain embodiments, the IDed substrate is linked to the oligonucleotide that is linked to the fluorophore. In certain embodiments, the IDed substrate is linked to the oligonucleotide that is linked to the quencher. In certain embodiments, the IDed substrate is linked to the fluorophore or the quencher.

In certain embodiments, the IDed substrate is a digitally coded bead. In certain embodiments, the IDed substrate is an ordered array. In certain embodiments, the IDed substrate comprises a colored quantum-dot.

In another aspect, the present disclosure provides a method for detecting multiple target nucleic acid sequences in a sample. In certain embodiments, the multiple target nucleic acid sequences comprise at least a first target sequence and a second target sequence. In certain embodiments, the method comprises the steps of: (a) contacting the sample with at least a first and a second IDed double-stranded probe as described herein, wherein the first IDed double-stranded probe comprises a sequence complementary to the first target sequence, and the second IDed double-stranded probe comprises a sequence complementary to the second target sequence, wherein the first IDed double-stranded probe comprises a first IDed substrate and the second IDed double-stranded probe comprises a second IDed substrate; (b) detecting a first fluorescence emitted by the first IDed double-stranded probe and a second fluorescence signal emitted by the second IDed double-stranded probe; and (c) analyzing the first and the second IDed substrate to determine the presence of the first and the second target sequence in the sample. In certain embodiments, the method further comprises the step of (d) analyzing the strength of the first and second fluorescence signal to determine the abundance of the first and the second target sequence.

In certain embodiments, the first target sequence and the second target sequence locate on a single nucleic acid. In certain embodiments, the first target sequence and the second target sequence locate on two separate nucleic acids.

In certain embodiments, the hybridization temperature ranges from 4° C.˜80° C. In certain embodiments, the hybridization temperature ranges from 4° C.˜70° C. In certain embodiments, the hybridization temperature ranges from 20° C.˜70° C. In certain embodiments, the hybridization temperature ranges from 20° C.˜50° C. In certain embodiments, the hybridization temperature ranges from 20° C.˜35° C. In certain embodiments, the hybridization temperature ranges from 20° C.˜30° C. In certain embodiments, the hybridization temperature is around 4° C., 6° C., 8° C., 10° C., 12° C., 14° C., 16° C., 18° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C. or 80° C.

In another aspect, the present disclosure provides a method for determining a sequence of a nucleic acid using a plurality of IDed double-stranded probes as described herein. In certain embodiments, the method comprises the steps of: (a) contacting the nucleic acid with at least a first and a second IDed double-stranded probe as described herein, wherein the first IDed double-stranded probe comprises a sequence complementary to the first target sequence, and the second IDed double-stranded probe comprises a sequence complementary to the second target sequence, the first IDed double-stranded probe comprises a first IDed substrate and the second IDed double-stranded probe comprises a second IDed substrate, wherein the first target sequence overlaps with the second target sequence; (b) detecting a first fluorescence emitted by the first IDed double-stranded probe and a second fluorescence signal emitted by the second IDed double-stranded probe; and (c) analyzing the first IDed substrate and the second IDed substrate to determine the first and the second target sequence; and (d) assembling the first and the second target sequence.

In yet another aspect, the present disclosure also provides a method for detecting a condition in a subject, comprising the steps of: (a) obtaining a sample to be tested from the subject; (b) contacting the sample with a plurality of IDed double-stranded probes as described herein; (c) detecting an IDed double-stranded probe that emits fluorescence; and (d) analyzing the IDed substrate of said detected IDed double-stranded probe to determine the presence of the condition in the subject.

In certain embodiments, the condition is selected from the group consisting of viral infection, cancer, a cardiac disease, a liver disease, a genetic disorder and an immunological disease.

In certain embodiments, the subject is a human.

In certain embodiments, the sample is selected from the group consisting of saliva, tears, blood, serum, urine, cell, and tissue biopsy.

### BRIEF DESCRIPTION OF THE FIGURES

FIGS. 1A-1B show a schematic illustration of an IDed double-stranded probe. In FIG. 1A, the IDed substrate is linked to the nucleotide. In FIG. 1B, the IDed substrate is linked to the fluorophore.

FIGS. 2A-2B show schematic illustration of the working principle of the IDed double-stranded probe. FIG. 2A illustrates the working principle of the spontaneous reaction between the IDed double-stranded probe with its single-strand target. FIG. 2B illustrates the working principle of the reaction between the IDed double-stranded probe with its double-stranded target during denaturation and annealing stage.

FIG. 3 shows schematic illustration of the working principle of the multiplex analysis using IDed double-stranded probes.

FIGS. 4A-4 shows schematic illustration of the working principle of sequencing a nucleic acid using IDed double-stranded probes.

### DETAILED DESCRIPTION OF THE INVENTION

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. In this disclosure, when a range is given as “(a first number) to (a second number)” or “(a first number)−(a second number),” this means a range whose lower limit is the first number and whose upper limit is the second number. For example, 2 to 10 nucleotides means a range whose lower limit is 2 nucleotides, and whose upper limit is 10 nucleotides.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, the embodiments described herein can be practiced without these specific details. In other instances, methods, procedures and components have not been described in detail so as not to obscure the related relevant function being described. Also, the description is not to be considered as limiting the scope of the implementations described herein. It will be understood that descriptions and characterizations of the embodiments set forth in this disclosure are not to be considered as mutually exclusive, unless otherwise noted.

### IDed Double-Stranded Probes

In one aspect, the present disclosure provides an IDed double-stranded probe comprising a double stranded nucleic acid hybridization probe associated with an IDed substrate that allows identification of the double-stranded probe. The double stranded nucleic acid hybridization probe consists of two complementary oligonucleotides of different lengths. One strand of the oligonucleotides is labeled with a fluorophore and the other is labeled with a quencher. The IDed double stranded probe can have different structures under different conditions, and this can be reflected by the fluorescence change. When self-hybridized in a stable double-stranded structure, the fluorophore and the quencher are in proximity, such that the fluorophore is quenched by the quencher, and the probe is non-fluorescent at the emission wavelength of the fluorophore. When under denatured conditions, such as in acid, basic or high temperature solution, the two strands of the probe are separated, and the fluorophore becomes fluorescent. In the presence of the target in hybridization solution, the longer strand of the probe can spontaneously bind to the target, the double-stranded probe becomes dissociated, and the fluorophore becomes fluorescent. When a plurality of IDed double-stranded probes exists in hybridization solution, the identity of the IDed double-stranded probe can be determined by detecting the IDed substrate associated with the double-stranded probe emitting fluorescence.

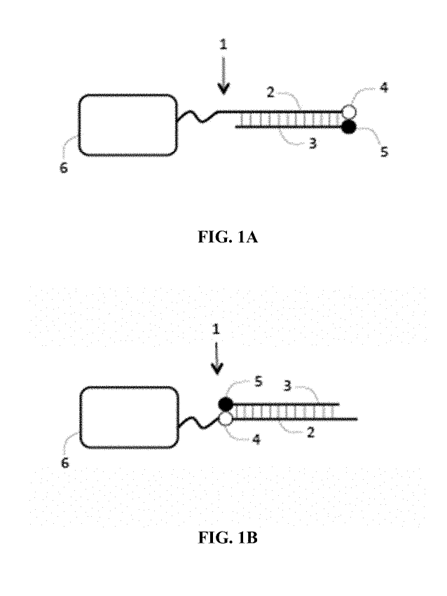

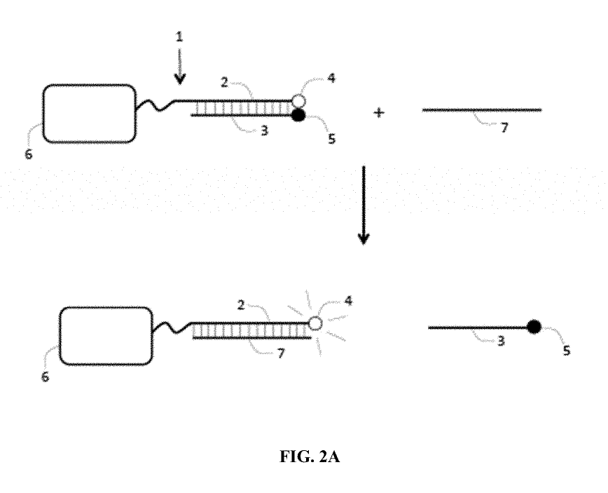

An exemplary embodiment of IDed double-stranded probes is illustrated in FIG. 1A. Referring to FIG. 1A, IDed double-stranded probe 1 is composed of two complementary oligonucleotides 2, 3 of different lengths. The longer strand, in this case positive strand 2, is labeled with a fluorophore 4 and an IDed substrate 6. The shorter negative strand 3 is labeled with a quencher 5. The probe is non-fluorescent due to the close proximity of the fluorophore and the quencher.

FIG. 1B illustrates another embodiment of IDed double-stranded probes. Referring to FIG. 1B, IDed double-stranded probe 1 is composed of two complementary oligonucleotides 2, 3 of different lengths. The longer strand 2, is labeled with a fluorophore 4 that is linked to an IDed substrate 6. The shorter negative strand 3 is labeled with a quencher 5. The probe is non-fluorescent due to the close proximity of the fluorophore and the quencher.

### Double-Stranded Probes

In certain embodiments, the oligonucleotide described above can comprise one or more nucleotide analogs (e.g., altered backbone, sugar, or nucleobase). In certain embodiments, the nucleotide analog is selected from the group consisting of 5-bromouracil, a peptide nucleic acid nucleotide, a xeno nucleic acid nucleotide, a morpholino, a locked nucleic acid nucleotide, a glycol nucleic acid nucleotide, a threose nucleic acid nucleotide, a dideoxynucleotide, a cordycepin, a 7-deaza-GTP, a fluorophore (e.g. rhodamine or flurescein linked to the sugar), a thiol containing nucleotide, a biotin linked nucleotide, a fluorescent base analog, a methyl-7-guanosine, a methylated nucleotide, an inosine, thiouridine, a pseudourdine, a dihydrouridine, a queuosine, and a wyosine. In certain embodiments, the nucleotide analog is a locked nucleic acid nucleotide.

In certain embodiments, the analog is a locked nucleic acid. A locked nucleic acid is a modified RNA nucleotide, in which the ribose moiety is modified with an extra bridge connecting the 2′ oxygen and 4′ carbon, thus locking the ribose in the 3′-endo conformation. The locked ribose conformation enhances base stacking and backbone pre-organization, which significantly increases the melting temperature of oligonucleotides.

In some embodiments, the length of the two strands ranges from 5-100 nucleotides, preferably 10-50 nucleotides, more preferably 15-25 nucleotides. In most cases, the two strands of the probes are different in length. In certain embodiments, the longer stand is 1-5 nucleotides longer than the shorter strand. In certain embodiments, the longer stand is 2-10, preferably 2-7, nucleotides longer than the shorter strand. In certain embodiments, the longer strand has a length of 5, 6, 7, 8, 9, 10, 11, 12, 13, 14, 15, 16, 17, 18, 19, 20, 21, 22, 23, 24, 25, 26, 27, 28, 29 or 30 nucleotides.

Suitable fluorophores and quenchers are exemplified without limitation in the compounds listed in Table 1 and Table 2. Suitable fluorophores and quenchers can be linked to the oligonucleotides using methods known in the art. For example, during synthesis of the oligonucleotide, phosphoramidite reagents containing protected flurophores, e.g., 6-FAM phosphoramidite, are reacted with hydroxyl groups to allow the preparation of flurophore-labeled oligonucleotide. Both fluorophore and the quencher can be linked on the terminal or internal bases of the double-stranded probes. In certain embodiments, they are on opposed terminal complementary bases of the two strands. In certain embodiments, both the fluorophore and the quencher are on the blunt end of the probe. In some cases, the position of the labels can be adjusted according the optimal quenching.

TABLE 1
Fluorophores for double-stranded probes

| | | Excitation | Emission |
|---|---|---|---|
| Fluorophore | Alternative Fluorophore | (nm) | (nm) |
| FAM | | 495 | 515 |
| TET | CAL Fluor Gold 540 | 525 | 540 |
| HEX | JOE, VIC, CAL Fluor Orange 560 | 535 | 555 |
| Cy3 | NED, Quasar 570, Oyster 556 | 550 | 570 |
| TMR | CAL Fluor Red 590 | 555 | 575 |
| ROX | LC red 610, CAL Fluor Red 610 | 575 | 605 |
| Texas red | LC red 610, CAL Fluor Red 610 | 585 | 605 |
| LC red 640 | CAL Fluor Red 635 | 625 | 640 |
| Cy5 | LC red 670, Quasar 670, Oyster | 650 | 670 |
| | 645 | | |
| LC red 705 | Cy5.5 | 680 | 710 |

TABLE 2
Quenchers for double-stranded probes

| Quencher | Absorption Maximum (nm) |
|---|---|
| DDQ-I | 430 |
| Dacyl | 475 |
| Eclipse | 530 |
| Iowa Black FQ | 532 |
| BHQ-1 | 534 |
| QSY-7 | 571 |
| BHQ-2 | 580 |
| DDQ-II | 630 |
| Iowa Black RQ | 645 |
| QSY-21 | 660 |
| BHQ-3 | 670 |

### IDed Substrate

“IDed substrate” as used herein, refers to a known code or a known label capable of generating a detectable signal that distinguishes one IDed substrate from another.

In certain embodiments, the IDed substrate is a digitally coded structure such as a digitally coded bead as described in U.S. Pat. No. 8,232,092 to Ho. Briefly, a digitally coded bead is a micro bead having a digitally coded structure that is partially transmissive and opaque to light and the pattern of transmitted light can be used to determine the identity of the bead. For example, the beads can comprise a body having a series of alternating light transmissive and opaque sections, with relative positions, widths and spacing resembling a 1D and 2D bar code image. To decode the image, the alternating transmissive and opaque sections of the body are scanned with light or imaged to determine the code represented by the image determined from the transmitted light. In certain embodiments, the digitally coded beads can be decoded using a microfluidic apparatus comprising a micro flow channel sized and configured to guide the coded beads to advance one at a time pass a decoding zone. The decoding zone includes a code detector that detects the pattern of transmitted light through each coded bead for decoding the code represented by the image.

In can be understood that the digitally coded structure as described above can be of any shape, such as rectangle, square, circle or oval, etc. Accordingly, the digital code can be of any form so long as it can generate distinguishable signal. For example, when the structure is a rectangular micro-plate, the digital code can be a bar-shape code. When the structure is a round micro-disc, the digital code can be a combination of certain patterns.

In certain embodiments, the IDed substrate is a multicolor semiconductor quantum-dot tagged bead as disclosed in Han et al, Nature Biotechnology, 19: 631-635 (2001) or U.S. patent application Ser. No. 10/185,226. Briefly, multicolor semiconductor quantum-dots are conjugated to or embedded in porous polymer beads. For each quantum-dot, there is a given intensity (with the levels of, for example, 0-10) and a given color (wavelength). For each single color coding, the porous polymer beads has different intensity of quantum-dots depending on the number of quantum-dots conjugated or embedded therein. If quantum-dots of multiple colors (n colors) and multiple intensity (m levels of intensity) are used, then the porous polymer beads may have a total number of unique identities or codes, which is equal to m to the exponent of n less one (mⁿ−1).

In certain embodiments, the IDed substrate is an ordered array. “Ordered array” as used herein, refers a solid surface on which a collection of double-stranded probes with known sequences are attached in an ordered manner, so that the identity (i.e., the sequences) of double-stranded probes can be determined based on their positions on the solid surface.

IDed substrate can be linked to nucleic acid through methods known in the art. For example, an oligonucleotide can be associated with the IDed substrate in non-covalent interactions (e.g., hydrogen bonds, ionic bonds, etc.) or covalent interactions. In certain embodiments, the oligonucleotide is associated to one or more functional groups on the substrate.

Any functional groups as disclosed herein can be used (e.g. amino, carboxyl, mercapto, phosphonate group, biotin, streptavidin, avidin, hydroxyl, alkyl or other molecules, linkers or groups). In certain embodiments, the nucleic acid is associated with the IDed substrate through streptavidin-biotin interactions. For example, the IDed substrate has streptavidin on its surface, and the nucleic acid is conjugated with biotin. After combining the two, streptavidin strongly binds to avidin and thereby associating the IDed substrate with the fragment of the nucleic acid.

### Spontaneous Reaction Between IDed Double-Stranded Probes with their Targets

IDed double-stranded probes having strands of different lengths can spontaneously react with single-stranded oligonucleotides comprising the target sequence in solution. In this reaction, the short strand in the double-stranded probe is displaced by the target oligonucleotide sequence to form a thermodynamically more stable duplex. The resulting dissociation of double-stranded probe produces an increase in fluorescence. In this reaction, easily designed embodiments of the double-stranded probes have the ability to distinguish perfectly matched targets from single-nucleotide mismatched targets at room temperature. This extremely high specificity lies in the fact that mismatched recognition is unfavored when compared with the self-reaction of the double strands of the probe itself. This is superior to single-stranded probes, because single-stranded probe are thermodynamically unstable, and can be hybridize with another single-stranded polynucleotide even there exists a mismatch. The same principle is exemplified in molecular beacons, which are more specific than linear probes due to their stable stem-loop structure that can out-compete a less stable mismatched reaction. However, the recognition portion of the molecular beacons, the loop, is still single-stranded, and this leaves room for mismatch hybridization, if the stem is not long enough or the loop sequence is too long. This is reflected by a recent report that molecular beacons cannot directly used for single-nucleotide discrimination when combined with NASBA (Nucleic Acid Sequence Based Amplification), a well-known isothermal nucleic acid amplification technique.

The IDed double-stranded probe can also be used to detect double-stranded nucleic acid comprising the target sequence. Typically, IDed double-stranded probes are mixed with double-stranded nucleic acids in a solution. The solution is heated to high-temperature (e.g., over 90° C., 95° C. or 98° C.), at which IDed double-stranded probes are denatured and dissociated. The solution is then cooled down to annealing temperature (e.g., about 40° C., 42° C. or 45° C.). In the absence of the target sequence, the two strands of the probe will be double-stranded conformation, and thus will be non-fluorescent. In the presence the target sequence, however, the two probe strands will hybridize with the target, which results in fluorescent. Alternatively, double stranded DNA can be denatured using alkaline buffer. For instance, the double stranded DNA can be mixed with denaturation buffer and incubated at certain temperature (e.g., around 50˜60° C.) for a period of time (e.g., about 5˜10 min). The neutralization buffer (e.g., NaAc) is then added before adding IDed probes for hybridization step.

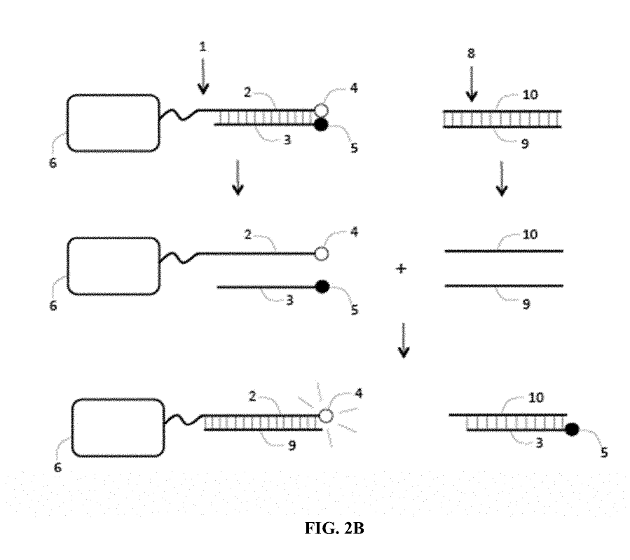

FIG. 2 illustrates an exemplary embodiment of spontaneous reaction between the IDed double-stranded probes with their targets. Referring to FIG. 2A, IDed double-stranded probe 1 is composed of two complementary oligonucleotides 2, 3 of different lengths. The longer strand 2 is labeled with a fluorophore 4 and an IDed substrate 6. The shorter negative strand 3 is labeled with a quencher 5. The probe is non-fluorescent due to the close proximity of the fluorophore and the quencher. In the presence of target 7, negative strand 3 is displaced by the target 7, and the escaped fluorophore 4 becomes fluorescent. It will be appreciated that fluorescence would also result, if fluorophore 4 and quencher 5 are interchanged. The IDed substrate 6 is detected and is used to determine the identity of the double-stranded probe 1.

Referring to FIG. 2B, there is shown a double-stranded probe 1 and a double-stranded nucleic acid 8. Probe 1 comprises strand 2, labeled with fluorophore 4 and an IDed substrate 6, and complementary strand 3, is labeled with quencher 5. The labels are applied to the blunt-end termini of the strands. Nucleic acid 8 comprises complementary strands 9, 10. Upon high-temperature denaturation, strands 2, 3 of the probe separate, as do strands 9, 10 of the nucleic acid. When the temperature is lowered to the annealing temperature, probe strands 2, 3 anneal, or hybridize, to their complementary target strands 9, 10 of the nucleic acid. Fluorophore 4 is not quenched by quencher 5, and fluoresces.

### IDed Double-Stranded Probes for Multiplexed Analysis

In another aspect, the present disclosure provides a method of detecting two or more different target sequences (either in different nucleic acids or in different portions of a given nucleic acid) simultaneously in a sample. The method involves using a set of IDed double-stranded probes, wherein each probe comprises a substrate of varying ID associated to a double-stranded probe with specific target sequence. Detection of the different target sequences in the sample arises from the combination of fluorophore emission and unique ID of the substrate.

In certain embodiments, a method of simultaneously detecting two or more different target sequences in a sample comprises (a) contacting the sample with two or more IDed double-stranded probes as described above, in which each probe comprises a different IDed substrate associated with a double-stranded probe that specifically binds to a different target sequence; (b) detecting IDed doube-stranded probes that emit fluorosence; and (c) analyzing said detected IDed substrate of the IDed double-stranded probes to determine the presence of the target sequences of the detected IDed double-stranded probes in the sample.

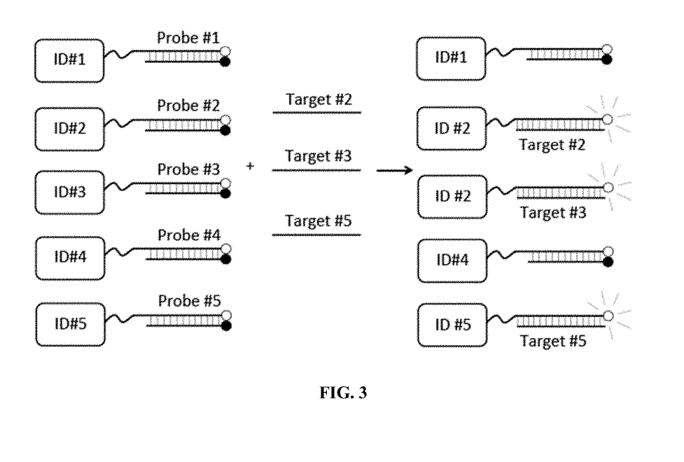

FIG. 3 illustrates an exemplary embodiment of the method for multiplexed analysis using IDed double-stranded probes. Referring to FIG. 3, five IDed double-stranded probes (Probes #1˜5) are contacted with a plurality of double-stranded nucleic acids, including Targets #2, 3 and 5, in one reaction. Each probe comprises a strand labeled with fluorophore and associated with an IDed substrate (ID #1˜5), and a complementary strand labeled with quencher. In the presence of Targets #2, 3 and 5, negative strands of Probes #2, 3 and 5 are displaced by the corresponding target, and the fluorophores of Probes #2, 3 and 5 are not quenched by quencher, and fluoresce. The IDed substrates of Probes #2, 3 and 5 are decoded to determine the identity (i.e., target sequence) of Probes #2, 3 and 5. The result indicates the presence of target sequences of Probes #2, 3 and 5, and absence of target sequences of Probes #1 and 4 in the nucleic acids. It is appreciated that Targets #2, 3, 5 may be multiple portions of a single target.

The method of detecting multiple targets (or multiple portions of a target) allows for a diagnostic library, wherein the library comprises multiple IDed double-stranded probes prepared as described above that flow through a microchannel or are spread on a substrate surface. The IDed double-stranded probes may or may not be chemically attached to the substrate surface. The IDed double-stranded probes can reside on the surface substrate through other non-bonding interactions (e.g., electrostatic interactions, magnetism, etc). The IDed double-stranded probes comprise double-stranded probes associated to IDed substrates through which the identities of the probes can be identified. The probes flow through a microchannel or are spread on a substrate surface by methods known in the art. The library can come in contact with a sample containing the target(s). After spontaneous reaction, the fluorescence emission will indicate which targets are present in the sample. Once a target is found to be present (or absent) in the sample, the identity of the probe will be determined through decoding the IDed substrate. By knowing the identities of the probes, the identity of the target sequence can be found. The diagnostic library can theoretically contain an unlimited number of conjugates. The diagnostic library will comprise at least one IDed double-stranded probe, preferably at least 20, 50, 100, 500, or 1000 probes.

### Sequencing of Nucleic Acid Using IDed Double-Stranded Probes

In another aspect, the present disclosure provides a method of sequencing a nucleic acid using IDed double-stranded probes as described above. In certain embodiments, the method comprises the step of hybridizing a group of IDed double-stranded probes to the nucleic acid, wherein the sequence of the nucleic acid can be assembled based on the sequences of the double-stranded probes.

As used herein, the term “nucleic acid” refers to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, robsomal RNA, ribozymes, cDNA, shRNA, single-stranded short or long RNAs, recombinant polynucleotides, branched poly-nucleotides, plasmids, vectors, isolated DNA of any sequence, control regions, isolated RNA of any sequence, nucleic acid probes, and primers. The nucleic acid may be linear or circular.

The assemble process can be accomplished based on at least two double-stranded probes whose sequences overlap. For example, in order to determine the sequence of a nucleic ace consisting contiguously of an upstream region, an overlap region and a down stream region, i.e., the 3′ end of the upstream region is linked to the 5′ end of the overlap region via a phosphodiester bond, and the 3′ end of the overlap region is linked to the 5′ end of the downstream region via a phosphodiester bond, at least two double-stranded probes are used. The first double-stranded probe is complementary to the first sequence consisting contiguously of the upstream region and an overlap region, and a second double-stranded probe is complementary to the second sequence consisting contiguously the overlap region and the downstream region. When the sequences of the first and the second double-stranded probes are determined, based on the overlap sequence (i.e., complementary to the overlap region), the sequence of the nucleic acid can be determined by assembling the sequences of the first and the second double-stranded probes. Similarly, when more probes hybridize to a nucleic acid and each of the probe overlaps with at least one other probe, the sequence of the nucleic acid can be determined by assembling the sequences of the probes. In certain embodiments, the upstream region has a length of 1, 2, 3, 4, 5, 6 or more nucleotides. In certain embodiments, the overlap region has a length of 3, 4, 5, 6, 7, 8, 9 or more nucleotides. In certain embodiments, the downstream region has a length of 1, 2, 3, 4, 5, 6 or more nucleotides.

Therefore, in certain embodiments, the method comprises (a) contacting the nucleic acid to be sequenced with a plurality of IDed double-stranded probes prepared as described above; (b) detecting a group of IDed double-stranded probes that emit fluorescence, wherein each probe in the group has a sequence overlapping with the sequence of at least one other probe in the group; (c) analyzing the IDed substrate of the IDed double-stranded probes detected to determine the sequence of each probe in the group; and (d) assembly the sequences of the probes in the group to determine the sequence of the nucleic acid.

In certain embodiments, the plurality of IDed double-stranded probes used to contact with the nucleic acid includes probes designed to represent a genomic regions of interest, preferably as large as an entire genome. In certain embodiments, each IDed double-stranded probe has a target sequence of X₁X₂X₃ . . . X_{N} (N=4-20), and wherein X can be any of A, T, C or G. And 4^{N} different IDed double-stranded probes may cover all permutations of n-mer oligonucleotides, thus represent an entire genome. For example, each IDed double-stranded probe has a target sequence of X₁X₂X₃X₄X₅X₆, wherein X can be any of A, T, C or G. As a result, 4⁶ (=4,096) different IDed double-stranded probes may cover all permutations of heptagon oligonucleotides, thus represent an entire genome. In other examples, each IDed double-stranded probe has a target sequence of X₁X₂X₃X₄X₅X₆X₇, X₁X₂X₃X₄X₅X₆X₇X₈, X₁X₂X₃X₄X₅X₆X₇X₈ X₉, or X₁X₂X₃X₄X₅X₆X₇X₈X₉X₁₀. wherein X can be any of A, T, C or G. Correspondingly, 4⁷, 4⁸, 4⁹ or 4¹⁰ different IDed double-stranded probes may cover all permutations of 7-mer, 8-mer, 9-mer or 10-mer oligonucleotides, thus represent an entire genome.

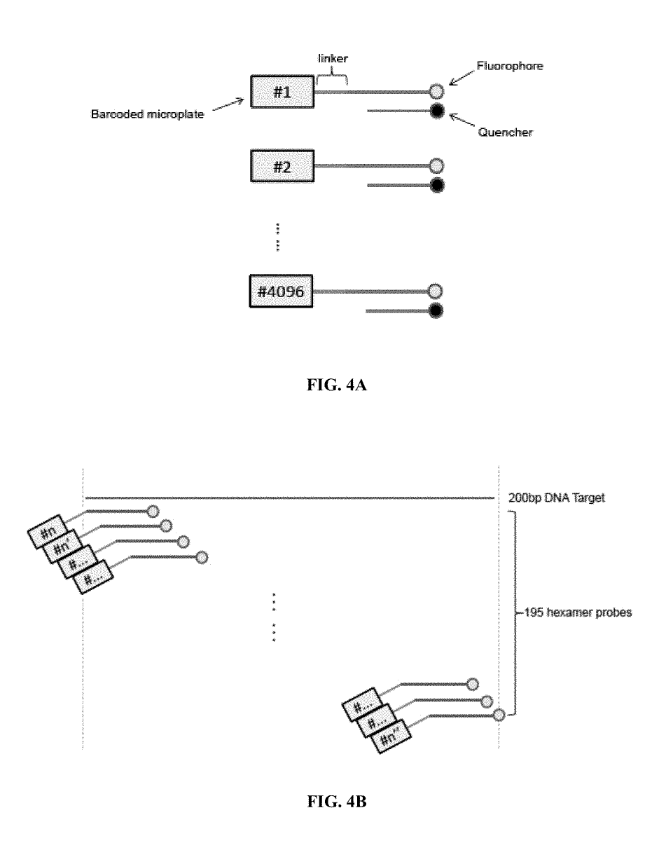

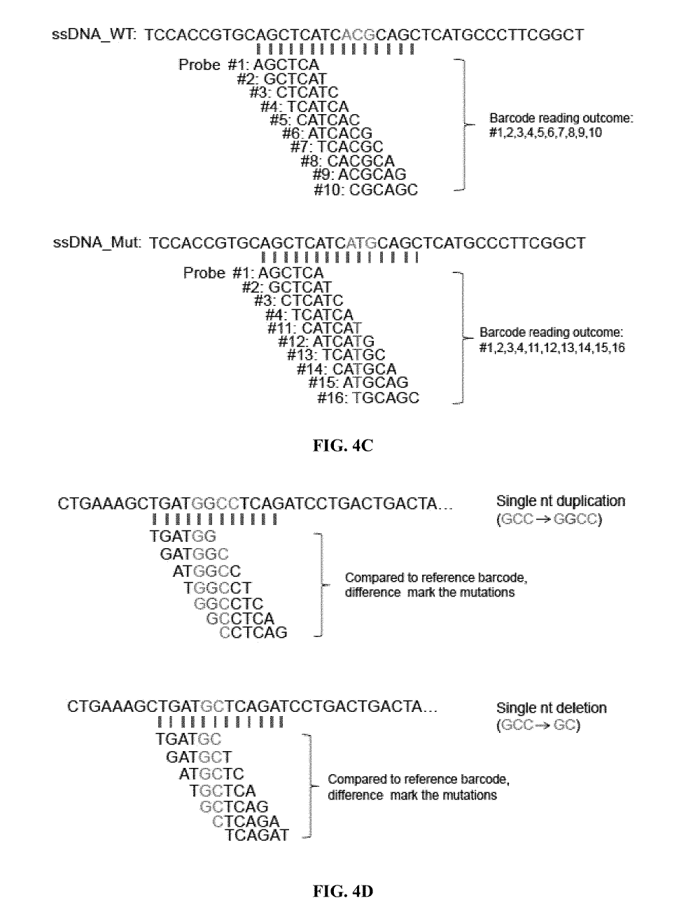

FIGS. 4A-4 illustrate an exemplary embodiment of methods for sequencing a nucleic acid.

Referring to FIG. 4A, a plurality of double-stranded probes are made of two complementary oligonucleotides of different length, the longer strand of which is a hexamer. Each IDed double-stranded probe has a target sequence of X₁X₂X₃X₄X₅X₆, wherein X can be any of A, T, C or G. As a result, 4⁶ (=4,096) different IDed double-stranded probes may cover all permutations of hexon oligonucleotides. The longer strand (hexamer) is labeled with a fluorophore and the shorter strand is labeled with a quencher. Each hexamer is linked with a barcoded microplate to distinguish it with other hexamers.

Referring to FIG. 4B, a given-length (x nt) DNA sequence (Target) can be assembled by x-5 DNA hexamer probes. In general, the Target DNA sequence is amplified through PCR and then mixed with 4,096 barcoded hexamer probes. The hexamer probes that hybridize with the DNA Target, thus emitting fluorescence are identified. The barcodes of the hexamer probes that emit fluorescence are read to determine the sequence of these hexamer probes. Alignment of all detected hexamer sequences allows assembling the DNA target sequence.

Referring to FIG. 4C, to demonstrate the utility of probes according to this invention in single-nucleotide mutation detection, two single DNA templates (targets) are prepared: a wildtype sequence ssDNA_WT (40 bp), which is used as reference and a sequence with one point mutation ssDNA_Mut (40 bp). The two targets differ from one another by a single nucleotide substitution.

After mixing the DNA template with the set of barcoded hexamer probes, the barcode microplate with fluorescent signaling are read and the hexamer sequences are assembled. In the wild-type reaction, fluorescence is detected in the probes with barcode#1, 2, 3, 4, 5, 6, 7, 8, 9, and 10, which corresponds with AGCTCA, GCTCAT, CTCATC, TCATCA, CATCAC, ATCACG, TCACGC, CACGCA, ACGCAG and CGCAGC, respectively. In the mutant reaction, fluorescence is detected in the probes with barcode#1, 2, 3, 4, 11, 12, 13, 14, 15 and 16, which corresponds with AGCTCA, GCTCAT, CTCATC, TCATCA, CATCAT, ATCATG, TCATGC, CATGCA, ATGCAG, and TGCAGC, respectively. After assembling the sequences of the detected probes, the sequences of the wild type and mutant DNA can be determined.

Referring to FIG. 4D, DNA sequences with single nucleotide duplication or single nucleotide deletion can be determined using the methods as described above.

In certain embodiments, in order to enrich the target sequence, the DNA template is amplified using PCR before mixing with the probes. In certain embodiments, an asymmetric PCR is used to generate single stranded Target sequences. As such, no denature step is required to detect the hybridization of the DNA template and the probes.

### Diagnosis of Diseases Using IDed Double-Stranded Probes

The present invention has applications in various diagnostic assays, including, but not limited to, the detection of viral infection, cancer, cardiac diseases, liver disease, genetic disorders and immunological diseases. The present invention can be used in a diagnostic assay to detect certain disease targets, by, for example, (a) obtaining a sample to be tested from a subject; (b) contacting the sample with a plurality of IDed double-stranded probes as described above, (c) detecting an IDed double-stranded probe that emits a fluorescence; (d) analyzing the IDed substrate of said detected IDed double-stranded probe to determine the presence of the condition in the subject. The sample of the subject can be bodily fluid, (e.g., saliva, tears, blood, serum, urine), cells, or tissue biopsy.

What is claimed is:

1. A method for detecting multiple target nucleic acid sequences in a sample, said multiple target nucleic acid sequences comprising at least a first target sequence and a second target sequence, said method comprising the steps of:
a) contacting the sample with at least a first IDed double-stranded probe and a second IDed double-stranded probe at a hybridization temperature,
wherein the first IDed double-stranded probe comprises:
(1) a first double-stranded nucleic acid hybridization probe consisting of
i) a first oligonucleotide comprising a first sequence complementary to the first target sequence;
ii) a second oligonucleotide comprising a second sequence that is complementary to the first sequence but is shorter than the first sequence by up to ten nucleotides;
iii) a first fluorophore linked to one of said first and second oligonucleotide; and
iv) a first fluorophore quencher linked to the other of said first and second oligonucleotide, wherein the first fluorophore quencher quenches the first fluorophore when the first oligonucleotide hybridizes to the second oligonucleotide; and
(2) a first IDed substrate associated with the first double-stranded nucleic acid hybridization probe;
wherein the second IDed double-stranded probe comprises:
(1) a second double-stranded nucleic acid hybridization probe consisting of
i) a third oligonucleotide comprising a third sequence complementary to the second target sequence;
ii) a fourth oligonucleotide comprising a fourth sequence that is complementary to the third sequence but is shorter than the third sequence by up to ten nucleotides;
iii) a second fluorophore linked to one of said third and fourth oligonucleotide; and
iv) a second fluorophore quencher linked to the other of said third and fourth oligonucleotide, wherein the second fluorophore quencher quenches the second fluorophore when the third oligonucleotide hybridizes to the fourth oligonucleotide; and
(2) a second IDed substrate associated with the double-stranded nucleic acid hybridization probe;
b) detecting a first fluorescence signal emitted by the first IDed double-stranded probe and a second fluorescence signal emitted by the second IDed double-stranded probes; and
c) analyzing the first IDed substrate and the second IDed substrate to determine the presence of the first and the second target sequences in the sample.

2. The method of claim 1, wherein said first oligonucleotide is capable of spontaneously hybridizing to said first target sequence in the presence of said second oligonucleotide and said third oligonucleotide is capable of spontaneously hybridizing to said second target sequence in the presence of said second oligonucleotide.

3. The method of claim 2, wherein said first oligonucleotide is not capable of spontaneously hybridizing to a mismatched sequence that differs from the first target sequence by a single nucleotide substitution and/or said third oligonucleotide is not capable of spontaneously hybridizing to a mismatched sequence that differs from the second target sequence by a single nucleotide substitution.

4. The method of claim 1, wherein said first and second oligonucleotides hybridize to produce a first double-stranded blunt end and said first fluorophore and said first quencher are linked to said first blunt end, and/or wherein said third and fourth oligonucleotides hybridize to produce a second double-stranded blunt end and said second fluorophore and said second quencher are linked to said second blunt end.

5. The method of claim 1, wherein each the first and the second target sequences has a length of 5˜30 nucleotides.

6. The method of claim 1, wherein said second oligonucleotide is shorter than said first sequence by 1 to 5 nucleotides and/or said fourth oligonucleotide is shorter than said third sequence by 1 to 5 nucleotides.

7. The method of claim 1, wherein said second oligonucleotide is shorter than said first sequence by 2 to 7 nucleotides and/or said fourth oligonucleotide is shorter than said third sequence by 2 to 7 nucleotides.

8. The method of claim 1, wherein said first sequence is 100% complementary to said first target sequence and/or said third sequence is 100% complementary to said second target sequence.

9. The method of claim 1, wherein the hybridization temperature ranges from 4° C.˜80° C.

10. The method of claim 1, wherein the first IDed substrate is linked to the oligonucleotide that is linked to the fluorophore and/or the first IDed substrate is linked to the oligonucleotide that is linked to the fluorophore.

11. The method of claim 1, wherein the first IDed substrate is linked to the oligonucleotide that is linked to the quencher and/or the second IDed substrate is linked to the oligonucleotide that is linked to the quencher.

12. The method of claim 1, wherein the first IDed substrate is linked to the fluorophore or the quencher and/or the second IDed substrate is linked to the fluorophore or the quencher.

13. The method of claim 1, wherein the first and/or the second IDed substrate is a digitally coded bead, an ordered array or a colored quantum-dot.

14. The method of claim 1, further comprising the step of (d) analyzing the strength of the first and second fluorescence signal to determine the abundance of the first and the second target sequence.

15. The method of claim 1, wherein the sample is derived from a patient, a livestock animal, environment or food.

16. A method of determining a sequence of a nucleic acid using a plurality of IDed double-stranded probes, wherein the sequence of the nucleic acid comprises contiguously an upstream sequence, an overlapping sequence and a downstream sequence, wherein the plurality of IDed double-stranded probes comprises at least a first IDed double-stranded probe and a second IDed double-stranded probe, wherein the first IDed double-stranded probe comprises:
(1) a first double-stranded nucleic acid hybridization probe consisting of
i) a first oligonucleotide comprising a first sequence complementary to a first target sequence consisting contiguously of the upstream sequence and the overlapping sequence;
ii) a second oligonucleotide comprising a second sequence that is complementary to the first sequence but is shorter than the first sequence by up to ten nucleotides;
iii) a first fluorophore linked to one of said first and second oligonucleotide; and
iv) a first fluorophore quencher linked to the other of said first and second oligonucleotide, wherein the first fluorophore quencher quenches the first fluorophore when the first oligonucleotide hybridizes to the second oligonucleotide; and
(2) a first IDed substrate associated with the first double-stranded nucleic acid hybridization probe;
wherein the second IDed double-stranded probe comprises:
(1) a second double-stranded nucleic acid hybridization probe consisting of
v) a third oligonucleotide comprising a third sequence complementary to a second target sequence consisting contiguously the overlapping sequence and the downstream sequence;
vi) a fourth oligonucleotide comprising a fourth sequence that is complementary to the third sequence but is shorter than the third sequence by up to ten nucleotides;
vii) a second fluorophore linked to one of said third and fourth oligonucleotide; and
viii) a second fluorophore quencher linked to the other of said third and fourth oligonucleotide, wherein the second fluorophore quencher quenches the second fluorophore when the third oligonucleotide hybridizes to the fourth oligonucleotide; and
(2) a second IDed substrate associated with the double-stranded nucleic acid hybridization probe;
the method comprising the steps of:
a) contacting the nucleic acid with the plurality of IDed double-stranded probes at a hybridization temperature;
b) detecting a first fluorescence signal emitted by the first IDed double-stranded probe and a second fluorescence signal emitted by the second IDed double-stranded probes; and
c) analyzing the first IDed substrate and the second IDed substrate to determine the first and the second target sequence; and
d) assembling the first and the second target sequence.

17. The method of claim 16, wherein said first oligonucleotide is capable of spontaneously hybridizing to said first target sequence in the presence of said second oligonucleotide and said third oligonucleotide is capable of spontaneously hybridizing to said second target sequence in the presence of said second oligonucleotide.

18. The method of claim 17, wherein said first oligonucleotide is not capable of spontaneously hybridizing to a mismatched sequence that differs from the first target sequence by a single nucleotide substitution and/or said third oligonucleotide is not capable of spontaneously hybridizing to a mismatched sequence that differs from the second target sequence by a single nucleotide substitution.

19. The method of claim 16, wherein each of the first and the second target sequences has a length of 5˜30 nucleotides.

20. The method of claim 19, wherein the hybridization temperature ranges from 4° C.˜80° C.


SEQUENCE LISTING (4 sequences)

SEQ ID NO: 1 - DNA, 40 bp, Artificial Sequence
  Synthetic
tccaccgtgcagctcatcacgcagctcatgcccttcggct

SEQ ID NO: 2 - DNA, 40 bp, Artificial Sequence
  Synthetic
tccaccgtgcagctcatcatgcagctcatgcccttcggct

SEQ ID NO: 3 - DNA, 34 bp, Artificial Sequence
  Synthetic
ctgaaagctgatggcctcagatcctgactgacta

SEQ ID NO: 4 - DNA, 32 bp, Artificial Sequence
  Synthetic
ctgaaagctgatgctcagatcctgactgacta